(19) United States
(12) Patent Application Publication
(10) Pub. No.: US 2024/0279266 A1
(43) Pub. Date: Aug. 22, 2024

(54) AN IMPROVED PROCESS FOR THE PREPARATION OF (4R)-1-[(2R,4R,5R)-3,3-DIFLUORO-4-HYDROXY-5-(HYDROXYMETHYL) OXOLAN-2-YL]-4-HYDROXY-1,3-DIAZINAN-2ONE AND ITS INTERMEDIATE COMPOUNDS

(71) Applicant: MSN LABORATORIES PRIVATE LIMITED, R&D CENTER, HYDERABAD, TELANGANA (IN)

(72) Inventors: Thirumalai Rajan SRINIVASAN, Hyderabad, Telangana (IN); Eswaraiah SAJJA, Hyderabad, Telangana (IN); Srinivas Reddy GADE, Hyderabad, Telangana (IN); Suresh CHALLA, Hyderabad, Telangana (IN); Shiva Shankar Goud GOPULARAM, Hyderabad, Telangana (IN)

(73) Assignee: MSN LABORATORIES PRIVATE LIMITED, R&D CENTER, Hyderabad, Telangana (IN)

(21) Appl. No.: 18/564,611

(22) PCT Filed: May 30, 2022
(86) PCT No.: PCT/IN2022/050500

§ 371 Date: Nov. 28, 2023

(30) Foreign Application Priority Data
May 29, 2021 (IN) 202141024011

(51) Int. Cl. C07H 19/06

(52) U.S. Cl. CPC C07H 19/06

(57) ABSTRACT

The present invention relates to an improved process for the preparation of (4R)-1-[(2R, 4R, 5R)-3,3-difluoro-4-hydroxy-5-(hydroxymethyl) oxolan-2-yl]-4-hydroxy-1,3-diazinan-2-one compound of formula-1 and its intermediate compounds. [Formula should be inserted here]. The present invention also relates to the process for the purification of (4R)-1-[(2R, 4R, 5R)-3,3-difluoro-4-hydroxy-5-(hydroxymethyl) oxolan-2-yl]-4-hydroxy-1,3-diazinan-2-one compound of formula-1 to get pure compound of formula-1.

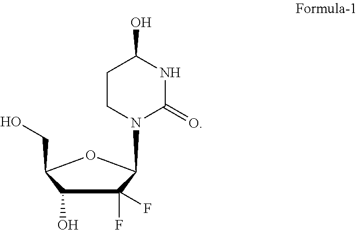

### RELATED APPLICATION

This application claims the benefit of priority to our Indian patent application number 202141024011 filed on May 29, 2021, the disclosures of all that are incorporated by reference in their entirety.

### FIELD OF THE INVENTION

The present invention relates to an improved process for the preparation of (4R)-1-[(2R,4R,5R)-3,3-difluoro-4-hydroxy-5-(hydroxymethyl) oxolan-2-yl]-4-hydroxy-1,3-diazinan-2-one compound of formula-1 and its intermediate compounds.

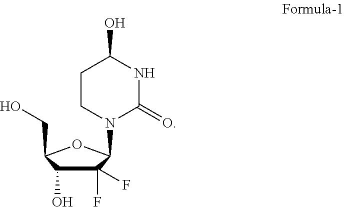

### BACKGROUND OF THE INVENTION

(4R)-1-[(2R,4R,5R)-3,3-difluoro-4-hydroxy-5-(hydroxymethyl) oxolan-2-yl]-4-hydroxy-1,3-diazinan-2-one compound of formula-1 is commonly known as Cedazuridine, which was approved in combination with Decitabine in US and Canada under the brand name of Ingovi for the treatment of adults with myelodysplastic syndromes (MDS) and chronic myelomonocytic leukemia (CMML).

Cedazuridine or pharmaceutically acceptable salts are described in U.S. Pat. No. 8,268,800B2. This patent also discloses the process for the preparation of Cedazuridine. The process as disclosed in the patent is schematically represented as below:

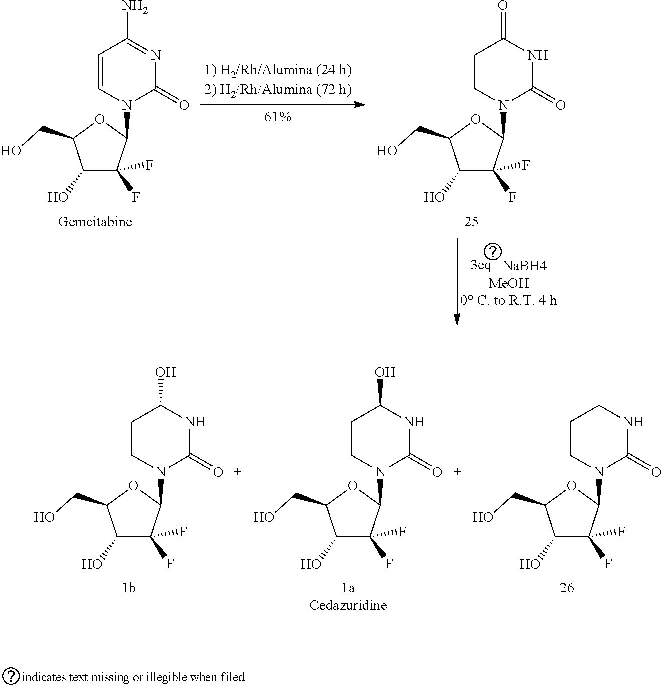

U.S. Pat. No. 9,567,363B2 patent describes the crystalline compound of Cedazuridine and also described its single crystal data.

U.S. Pat. No. 9,834,576B2 describes the process for the preparation of Cedazuridine compound of formula-1 and its crystallization method. The process steps as illustrated by the following scheme:

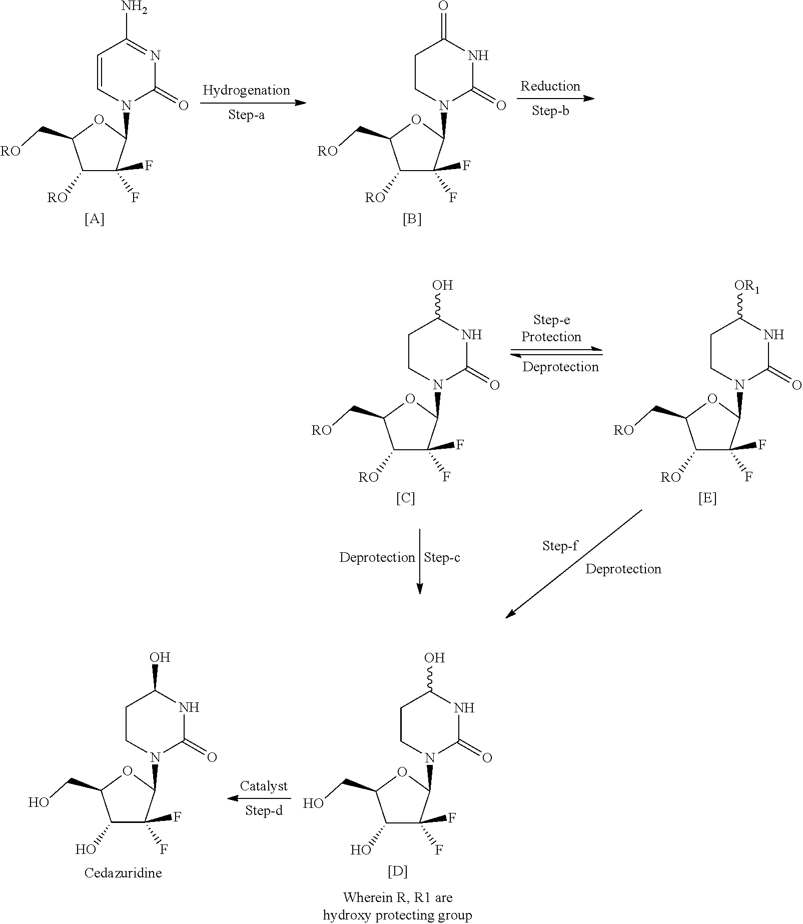

The present inventors developed the isolation of pure Cedazuridine from the mixture without using catalyst.

Journal of Medicinal Chemistry, 2014, 57(6), 2582-2588 discloses the process for the preparation of Cedazuridine as depicted below:

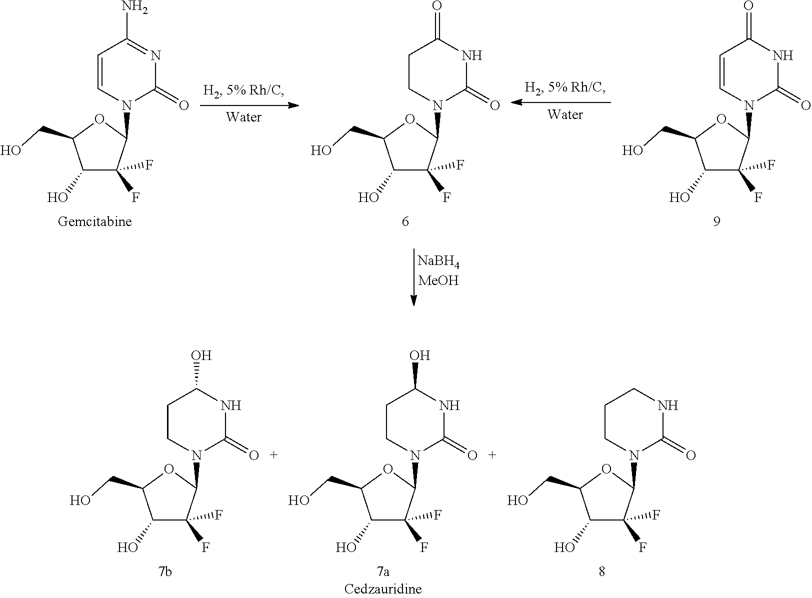

However, there is always a need exist for alternative preparative routes, which for example involves fewer steps, use reagents that are less expensive and/or easier to handle, consumes smaller amounts of reagents, provide a higher yield of product, have smaller and/or more ecofriendly waste products, and/or provide a higher purity of the final compound of Formula-1. Hence the inventors of the present invention have developed an improved process for the preparation of Cedazuridine and its intermediate compounds.

### OBJECTIVE OF INVENTION

An objective of the present invention is to provide commercially viable and cost-effective improved processes for the preparation of Cedazuridine of formula-1 and its intermediate compounds with high purity.

An another objective of the present invention is to provide catalyst free purification process for the preparation of pure Cedazuridine with desired epimer.

### SUMMARY OF THE INVENTION

The present invention relates to an efficient method for synthesizing Cedazuridine and its intermediates.

The first embodiment of the present invention is to provide the process for the preparation of Cedazuridine comprising the steps of:
a) reduction of 4-Amino-1-((4R,5R)-3,3-difluoro-4-hydroxy-5-(hydroxymethyl) tetrahydrofuran-2-yl) pyrimidin-2(1H)-one or its hydrochloride to get the compound of Formula-3,
b) reduction of compound of Formula-3 in presence of catalyst and reducing agent in a solvent to give the mixture of Cedazuridine of formula-1 and its epimer,
c) isolating pure Cedazuridine of Formula-1 from the compound obtained in step-b).

Second embodiment of the present invention is to provide the process for the preparation of Cedazuridine of formula-1 comprising the steps of.
a) protecting the 4-Amino-1-((4R,5R)-3,3-difluoro-4-hydroxy-5-(hydroxymethyl) tetrahydrofuran-2-yl) pyrimidin-2(1H)-one compound of Formula-2 or its hydrochloride salt to get compound of Formula-4;
b) reacting the compound of Formula-4 with an acid in a solvent to get the compound of Formula-5;
c) deprotecting the compound of Formula-5 with the deprotecting agent in presence of solvent to get the compound of Formula-6;
d) hydrogenating the compound obtained in step-c in presence of reducing agent in a solvent to get the compound of Formula-3;
e) converting the compound of formula-3 into Cedazuridine of Formula-1.

The third embodiment of the present invention provides process for the preparation of substantially pure Cedazuridine compound of formula-1.

### BRIEF DESCRIPTION ON DRAWINGS

FIG. 1: Illustrates PXRD [Powder X-Ray Diffraction] pattern of Crystalline form of Cedazuridine of formula-1.

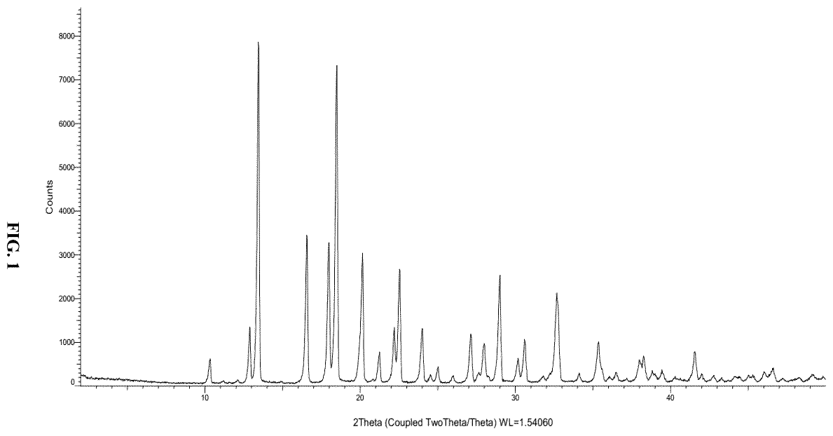

### DETAILED DESCRIPTION

The “solvent” used in the present invention is selected from but not limited to “hydrocarbon solvents” such as n-pentane, n-hexane, n-heptane, cyclohexane, petroleum ether, benzene, toluene, xylene and mixtures thereof; “ether solvents” such as dimethyl ether, diethyl ether, diisopropyl ether, methyl tert-butyl ether, 1,2-dimethoxyethane, tetrahydrofuran, 1,4-dioxane and mixtures thereof; “ester solvents” such as methyl acetate, ethyl acetate, n-propyl acetate, isopropyl acetate, n-butyl acetate, isobutyl acetate, tert-butyl acetate and mixtures thereof; “polar-aprotic solvents” such as dimethylacetamide, dimethylformamide, dimethylsulfoxide, N-methylpyrrolidone (NMP) and mixtures thereof; “chloro solvents” such as dichloromethane, dichloroethane, chloroform, carbon tetrachloride and mixtures thereof; “ketone solvents” such as acetone, methyl ethyl ketone, methyl isobutyl ketone and mixtures thereof; “nitrile solvents” such as acetonitrile, propionitrile, isobutyronitrile and mixtures thereof; “alcohol solvents” such as methanol, ethanol, n-propanol, iso-propanol, n-butanol, iso-butanol, 2-butanol, tert-butanol, ethane-1,2-diol, propane-1,2-diol and mixtures thereof; “polar solvents” such as water; formic acid, acetic acid and the like or mixture of any of the afore mentioned solvents.

The term “base” used in the present invention refers to inorganic bases selected from “alkali metal carbonates” such as sodium carbonate, potassium carbonate, lithium carbonate, cesium carbonate and the like; “alkali metal bicarbonates” such as sodium bicarbonate, potassium bicarbonate, lithium bicarbonate, cesium bicarbonate and the like; “alkali metal hydroxides” such as sodium hydroxide, potassium hydroxide, lithium hydroxide and the like; “alkyl metals” such as n-butyl lithium and like; “metal hydrides” such as lithium hydride, sodium hydride, potassium hydride and the like; “alkali metal phosphates” such as disodium hydrogen phosphate, dipotassium hydrogen phosphate; ammonia such as aqueous ammonia, ammonia gas, methanolic ammonia and like and “organic bases” selected from but not limited to methyl amine, ethyl amine, diisopropyl amine, diisopropylethyl amine (DIPEA), diisobutylamine, triethylamine, tert·butyl amine, pyridine, 4-dimethylaminopyridine (DMAP), N-methyl morpholine (NMM), n-methyl pyridine (NMP), 1,8-diazabicyclo[5.4.0]undec-7-ene (DBU), 1,5-diazabicyclo [4.3.0]non-5-ene (DBN), 1,4-diazabicyclo[2.2.2]octane (DABCO), imidazole; “alkali metal alkoxides” such as sodium methoxide, sodium ethoxide, potassium methoxide, potassium ethoxide, sodium tert·butoxide, potassium tert·butoxide and the like; “alkali metal amides” such as sodium amide, potassium amide, lithium amide, lithium diisopropyl amide (LDA), sodium bis(trimethylsilyl)amide (NaHMDS), potassium bis(trimethylsilyl)amide, lithium bis(trimethysilyl)amide (LiHMDS) and the like; or mixtures thereof.

As used herein the term “acid” in the present invention refers to inorganic acid and organic acid; inorganic acid is selected from such as hydrochloric acid, hydrobromic acid, sulfuric acid, nitric acid, phosphoric acid, sulfuric acid; organic acids such as acetic acid, maleic acid, malic acid, oxalic acid, succinic acid, fumaric acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (IS)-(+)-camphor sulfonic acid (1R)-(+)-bromocamphor-10-sulfonic acid, (1S)-(−)-bromocamphor-10-sulfonic acid, (−)-Dibenzoyl-L-tartaric acid, (−)-Dibenzoyl-L-tartaric acid monohydrate, (+)-Dibenzoyl-D-tartaric acid, (+)-Dibenzoyl-D-tartaric acid monohydrate, (+)-dipara-tolyl-D-tataric acid, (−)-dipara-tolyl-L-tataric acid, L(−)-pyroglutamic acid, L(+)-pyroglutamic acid, (−)-lactic acid; or chiral amino acid selected from but not limited to D-isomers and L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, norleucine, norvaline, hydroxyproline, sarcosine, citrulline, cysteic acid, t-butylglycine, t-butylalanine, phenylglycine, cyclohexylalanine, N-acetyl-leucine etc., and like

As used herein the term “Protecting agent” used in the present invention is selected from but not limited to alkyl, cycloalkyl, arylalkyl, aryl, ethers, esters, cyclic ethers, cyclic esters, acetal, cyclic acetal, ketal, and cyclic ketal groups and the like that can be removed under either acidic or basic conditions so that the protecting group is removed and replaced with a hydrogen atom. Specific hydroxyl protecting groups include, but are not limited to, methyl, ethyl, acetate, ethylacetate, propionate, ethylene glycol, propylene glycol, 4-methoxybenzyl, benzyl, trityl, trimethylsilyl, tetrahydropyranyl, and benzoyl.

As used herein the term “Deprotecting agent” used in the present invention is selected from but not limited to mild base in presence of methanol selected from ammonia, trimethyl ammonia, pyridine, ammonium hydroxide, KCN and methanol and hydrochloric acid, hydrobromic acid, hydrofluoric acid, sulfuric acid, nitric acid, phosphoric acid, acetic acid, trifluoroacetic acid, formic acid, substituted/unsubstituted alkyl/aryl sulfonic acids such as methanesulfonic acid, ethanesulfonic acid, benzenesulfonic acid, p-toluenesulfonic acid, pyridinium p-toluene sulfonic acid, trifluromethane sulfonic acid and “hydrogen fluoride (HF) sources” such as ammonium fluoride, tetrabutyl ammonium fluoride, pyridine-HF, Et₃N-3HF etc; metal catalysts in presence of hydrogen source and the like.

As used herein the term “Reducing agent” used in the present invention is selected from but not limited to Metal hydride including but not limited to LiAlH₄, NaAlH₄, NaBH₄, KBH₄, mixture of NaBH₄ & acetic acid, mixture of NaBH₄ & trifluoroacetic acid, mixture of NaBH₄ & iodine, mixture of NaBH₄ & trimethylchlorosilane, mixture of NaBH4 & magnesium chloride, mixture of NaBH₄ & calcium chloride, mixture of NaBH4 & one of transition metal chlorides, mixture of sodium borohydride BF₃·etherate, sodium cyanoborohydride, sodium triacetoxy borohydride, Aluminium hydride (AlH₃), diisobutylaluminium hydride (DIBAL), Vitride {=Sodium bis(2-methoxyethoxy) aluminum hydride}, Lithium Tri-tert-butoxyaluminum Hydride, Tributyltin Hydride; boranes such as not limited to BH₃-tetrahydrofuran, BH₃-dimethyl sulfide; hydrazine; metal including but not limited to Na, Fe, Ni, Zn, Sn in presence of acidic medium; Na-liquid ammonia; Silanes including but not limited to tri(C1-C6)alkylsilanes, tri(C1-C6)alkylsilyl halides; catalytic hydrogenation in presence of transition metals catalysts including but not limited to Ni, Pd, Pt, Rh, Re, Ru and Ir, including their oxides, hydroxides, acetates and combinations and the like.

As used herein the term “Catalyst” used in the present invention is selected from but not limited to metal catalysts Cerium Chloride and its heptahydrate.

The “room temperature” or “ambient temperature” used in the present invention is the temperature about 25° C.-35° C.

The term “pure” or “substantially pure” in the present invention refers to Cedazuridine of formula-1 is having purity greater than about 95%, greater than about 97%, greater than about 99% by HPLC {High Performance Liquid Chromatography}.

“Cedazuridine of formula-1” or “Isomeric mixture of Cedazuridine of formula-1” or “mixture of Cedazuridine of formula-1 and it's epimer” in the present invention refers to the mixture contains 4S and 4R in the ratio of about 50:50 to about 1:99.

The processes for the preparation of Cedazuridine of present application are schematically represented below in Schemes I & II:

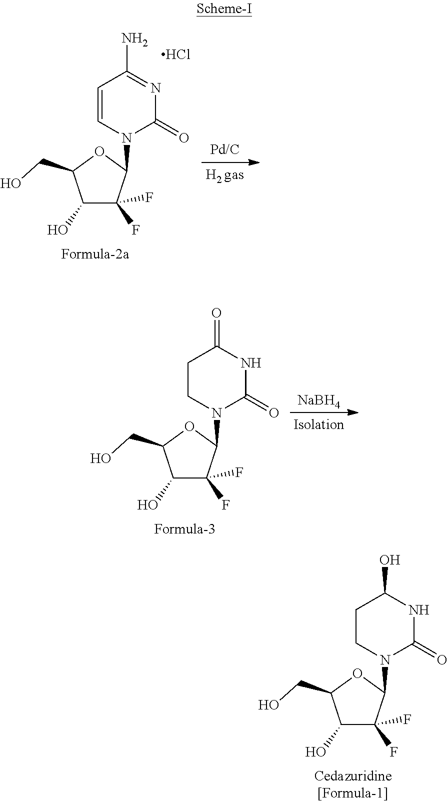

The first embodiment of present invention provides an improved process for the preparation of Cedazuridine of formula-1 comprises:
a) reduction of 4-Amino-1-((4R,5R)-3,3-difluoro-4-hydroxy-5-(hydroxymethyl) tetrahydrofuran-2-yl) pyrimidin-2(1H)-one compound of formula-2 or its hydrochloride compound of Formula-2a with a reducing agent in presence hydrogen gas in a solvent to get the compound of Formula-3,
b) reduction of the compound of Formula-3 with reducing agent, in presence of a catalyst and in a solvent to get the mixture of Cedazuridine of formula-1 and its epimer,
c) isolating pure Cedazuridine of Formula-1 from the compound obtained in step-b).

In first aspect of the first embodiment, the reducing agent used in step-a) is selected from but not limited catalytic hydrogenation in presence of transition metals catalysts including but not limited to Ni, Pd, Pt, Rh, Re, Ru and Ir, including their oxides, hydroxides, acetates and combinations and the like.

In second aspect of the first embodiment, the solvents used in step-a) and step-b) are selected from but not limited to hydrocarbon solvents, ether solvents, ester solvents, polar-aprotic solvents, chloro solvents, ketone solvents, nitrile solvents, alcohol solvents, polar solvents such as water and/or their mixtures thereof as described above.

In third aspect of the first embodiment, the reducing agent in step-b) is selected from “reducing agent” as defined above; and Cerium Chloride or Cerium Chloride heptahydrate.

In fourth aspect of the first embodiment, isolation of pure Cedazuridine of formula-1 in step-c) can be carried out by known techniques such as fractional distillation, chromatographic techniques or crystallization techniques and like.

In fifth aspect of the first embodiment, wherein the isolation of pure Cedazuridine in step-c) can be carried out by suspending the compound obtained in step-b) in a solvent followed by decanting or filtering the mixture.

In sixth aspect of the third embodiment, the isolation of pure Cedazuridine in step-c) can be carried without using any catalyst for getting enhanced production of the desired epimer. Such catalysts are selected from an acid, e.g., an inorganic acid, e.g., an organic acid, e.g., acetic acid or trifluoroacetic acid and a base, e.g., a Bronsted-Lowry base; base has a pKa of 10 or more i.e., DMSO; DBU {1,8-diazabicyclo(5.4.0)undec-7-ene}.

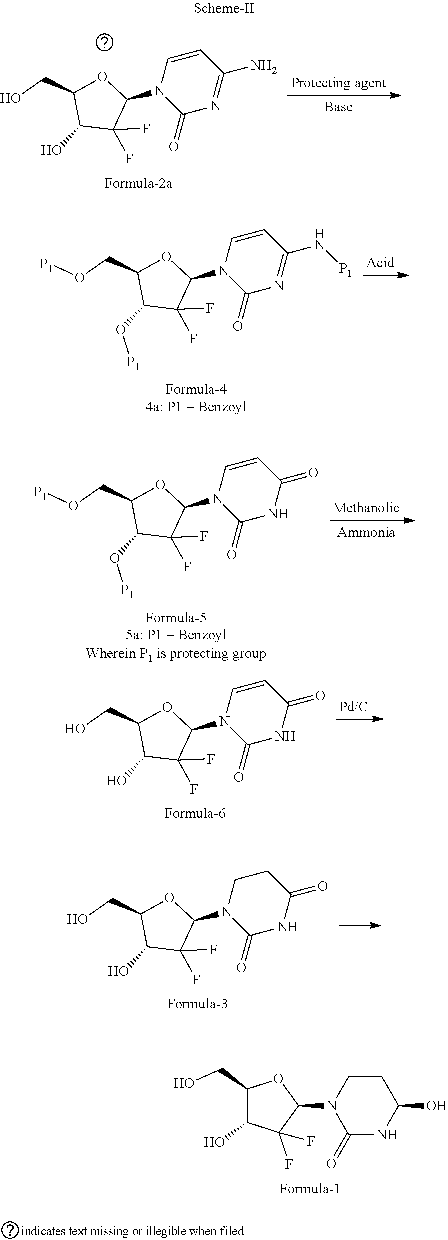

The second embodiment of present invention provides a process for the preparation of Cedazuridine of formula-1 comprises:
a) protecting 4-Amino-1-((4R,5R)-3,3-difluoro-4-hydroxy-5-(hydroxymethyl) tetrahydrofuran-2-yl) pyrimidin-2(1H)-one compound of Formula-2 or its hydrochloride compound of Formula-2a using protecting agent in presence of base in a solvent to get compound of Formula-4;
b) reacting compound of Formula-4 obtained in step-a with an acid in a solvent to provide compound of Formula-5;
c) deprotecting the compound of Formula-5 with a deprotecting agent in a solvent to provide compound of Formula-6;
d) hydrogenating the compound obtained in step-c) in presence of reducing agent in a solvent to get the compound of Formula-3;
e) converting the compound of formula-3 into Cedazuridine of Formula-1.

The protecting agent used in step-a) is selected from alkyl, cycloalkyl, arylalkyl, aryl, ethers, esters, cyclic ethers, cyclic esters, acetal, cyclic acetal, ketal, and cyclic ketal groups and the like that can be removed under either acidic or basic conditions so that the protecting group is removed and replaced with a hydrogen atom. Specific hydroxyl protecting groups include, but are not limited to, methyl, ethyl, acetate, ethylacetate, propionate, ethylene glycol, propylene glycol, 4-methoxybenzyl, benzyl, trityl, trimethylsilyl, tetrahydropyranyl, and benzoyl.

The base used in step-a) is selected from inorganic base selected from sodium carbonate, potassium carbonate, lithium carbonate, cesium carbonate or sodium bicarbonate, potassium bicarbonate, lithium bicarbonate, cesium bicarbonate and the like or sodium hydroxide, potassium hydroxide, lithium hydroxide and the like or n-butyl lithium or lithium hydride, sodium hydride, potassium hydride and the like or organic bases selected from methyl amine, ethyl amine, diisopropyl amine, diisopropylethyl amine (DIPEA), diisobutylamine, triethylamine, tert·butyl amine, pyridine, 4-dimethylaminopyridine (DMAP), N-methyl morpholine (NMM), n-methyl pyridine (NMP), 1,8-diazabicyclo[5.4.0]undec-7-ene (DBU), 1,5-diazabicyclo [4.3.0]non-5-ene (DBN), 1,4-diazabicyclo[2.2.2]octane (DABCO), imidazole; “alkali metal alkoxides” such as sodium methoxide, sodium ethoxide, potassium methoxide, potassium ethoxide, sodium tert·butoxide, potassium tert·butoxide and the like; “alkali metal amides” such as sodium amide, potassium amide, lithium amide, lithium diisopropyl amide (LDA), sodium bis(trimethylsilyl)amide (NaHMDS), potassium bis(trimethylsilyl)amide, lithium bis(trimethysilyl)amide (LiHMDS) and the like; or mixtures thereof.

The acid used in step-b) is selected from inorganic acid and organic acid; inorganic acid is selected from such as hydrochloric acid, hydrobromic acid, sulfuric acid, nitric acid, phosphoric acid, sulfuric acid; organic acids such as acetic acid, maleic acid, malic acid, oxalic acid, succinic acid, fumaric acid, trifluoroacetic acid, methane sulfonic acid, p-toluene sulfonic acid; chiral acids such as S-(+) mandelic acid, R-(−) mandelic acid, L-(+)tartaric acid, D-(−)tartaric acid, L-malic acid, D-malic acid, D-maleic acid, (−)-naproxen, (+)-naproxen, (1R)-(−)-camphor sulfonic acid, (IS)-(+)-camphor sulfonic acid (1R)-(+)-bromocamphor-10-sulfonic acid, (1S)-(−)-bromocamphor-10-sulfonic acid, (−)-Dibenzoyl-L-tartaric acid, (−)-Dibenzoyl-L-tartaric acid monohydrate, (+)-Dibenzoyl-D-tartaric acid, (+)-Dibenzoyl-D-tartaric acid monohydrate, (+)-dipara-tolyl-D-tataric acid, (−)-dipara-tolyl-L-tataric acid, L(−)-pyroglutamic acid, L(+)-pyroglutamic acid, (−)-lactic acid; or chiral amino acid selected from but not limited to D-isomers and L-isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-amino isobutyric acid, 3-amino propionic acid, norleucine, norvaline, hydroxyproline, sarcosine, citrulline, cysteic acid, t-butylglycine, t-butylalanine, phenylglycine, cyclohexylalanine, N-acetyl-leucine etc., and the like as described above.

The deprotecting agent used in step-c) is selected from mild base in presence of methanol such as ammonia, trimethyl ammonia, pyridine, ammonium hydroxide, KCN and methanol and hydrochloric acid, hydrobromic acid, hydrofluoric acid, sulfuric acid, nitric acid, phosphoric acid, acetic acid, trifluoroacetic acid, formic acid, substituted/unsubstituted alkyl/aryl sulfonic acids such as methanesulfonic acid, ethanesulfonic acid, benzenesulfonic acid, p-toluenesulfonic acid, pyridinium p-toluene sulfonic acid, trifluromethane sulfonic acid and “hydrogen fluoride (HF) sources” such as ammonium fluoride, tetrabutyl ammonium fluoride, pyridine-HF, Et3N-3HF etc; metal catalysts in presence of hydrogen source and the like.

The reducing agent in step-d) is selected from “reducing agent” as defined above.

The third embodiment of the present invention provides substantially pure Cedazuridine of formula-1 comprises:
a) dissolving or suspending Cedazuridine of formula-1 or isomeric mixture of Cedazuridine of formula-1 in a solvent,
b) isolating pure Cedazuridine of formula-1 from the mixture obtained in step-a).

In first aspect of the third embodiment, the solvent in step-a) selected from nitrile solvents, alcohol solvents, ketone solvents, polar aprotic solvents and water and/or mixtures thereof.

In second aspect of the third embodiment, isolation of pure Cedazuridine of formula-1 in step-b) is by solvent removal by known techniques, which are selected from drying, decanting, filtration, cooling the mixture to lower temperatures to precipitate the solid followed by filtration of the mixture, crystallization or crystallization by adding an anti-solvent.

In third aspect of the third embodiment, the isolation of pure Cedazuridine can be carried without using any catalyst for getting enhanced production of the desired epimer. Such catalysts are selected from an acid, e.g., an inorganic acid, e.g., an organic acid, e.g., acetic acid or trifluoroacetic acid and a base, e.g., a Bronsted-Lowry base; base has a pKa of 10 or more i.e., DMSO; DBU.

Purity of Cedazuridine of formula-1 and its intermediates can be analyzed by known HPLC {High Performance Liquid Chromatography} or GC {Gas chromatography} methods.

In fourth embodiment, PXRD [Powder X-Ray Diffraction] pattern of Cedazuridine of formula-1 obtained according to the present invention is illustrated in FIG.-1.

The fifth embodiment of present invention presents the intermediates obtained as per Scheme-I and Scheme-II of the present invention are useful in the preparation of Cedazuridine of formula-1.

Advantageously, the process of present invention provides substantially pure Cedazuridine of formula-1 with good yields and purity.

The present invention provides simple, economical, ecofriendly, reproducible, robust process for the preparation of Cedazuridine of formula-1, which is well feasible on a commercial scale.

Cedazuridine compound of formula-1 prepared according to the present invention can be further micronized or milled in conventional techniques to get the desired particle size to achieve desired solubility profile based on different forms of pharmaceutical composition requirements. Techniques that may be used for particle size reduction include, but not limited to ball milling, roll milling and hammer milling, and jet milling. Milling or micronization may be performed before drying, or after the completion of drying of the product.

The sixth embodiment of the present invention provides pharmaceutical composition comprising Cedazuridine of formula-1 and one or more pharmaceutically acceptable excipients.

Wherein, the pharmaceutically acceptable excipients selected from but not limited to binders, diluents, disintegrants, surfactants and lubricants; the binders that can be include polyvinylpyrolidone, copovidone, starches such as pregelatinized starch, cellulose derivatives such as hydroxypropylmethyl cellulose, ethylcellulose, hydroxypropylcellulose and carboxymethylcellulose, gelatine, acacia, agar, alginic acid, carbomer, chitosan, dextrates, cyclodextrin, dextrin, glycerol dibehenate, guargum, hypromellose, maltodextrin, poloxamer, polycarbophil, polydextrose, polyethylene oxide, polymethacrylates, sodium alginate, sucrose, mixtures thereof, the diluents that can be include anhydrous lactose, lactose monohydrate, modified lactose, dibasic calcium phosphate, tribasic calcium phosphate, microcrystalline cellulose, silicified microcrystalline cellulose, powdered cellulose, maize starch, pregelatinized starch, calcium carbonate, sucrose, glucose, dextrates, dextrins, dextrose, fructose, lactitol, mannitol, sorbitol starch, calcium lactate or mixtures thereof; the disintegrants that can be include magnesium aluminometa silicate (or magnesium aluminum silicate), starch, pregelatinized starch, sodium starch glycolate, crospovidone, croscarmellose sodium, low-substituted hydroxypropyl cellulose, alginic acid, carboxy methyl cellulose sodium, sodium alginate, calcium alginate and chitosan; the lubricants that can be include (but are not limited to) magnesium stearate, stearic acid, palmitic acid, talc, and aerosol; the surfactants that can be included (but are not limited to) polysorbate 80, polyoxyethylene sorbitan, polyoxyethylene-polyoxy-propylene copolymer and sodium lauryl sulphate; beta-cyclodextrin includes (but are not limited to) sulfobutylalkyl ether-beta-cyclodextrin, betadex-sulfobutylether sodium, or hydroxypropyl-beta-cyclodextrin.

As used herein, the term “pharmaceutical composition” or “pharmaceutical formulation” is formulated in a manner suitable for the route of administration to be used that include tablets, pills, powders, liquids, suspensions, emulsions, granules, capsules, suppositories, or injection preparations.

One another embodiment of the present invention provides a pharmaceutical composition comprising Cedazuridine of formula-1 prepared according to the present invention in combination with Decitabine or Azacitidine and one or more pharmaceutically acceptable excipients used for the treatment of adult patients with myelodysplastic syndromes (MDS) and chronic myelomonocytic leukemia (CMML).

Another embodiment of the present invention provides a pharmaceutical composition comprising Cedazuridine of formula-1 prepared according to the present invention in combination with Azacitidine and one or more pharmaceutically acceptable excipients used for the treatment.

The present invention is illustrated with the following examples that are provided by way of illustration only and should not be construed to limit the scope of invention in any manner whatsoever.

### EXAMPLES

### Example-1: Preparation of Compound of Formula-4a

Mixture of Gemcitabine hydrochloride (50 g) of compound of formula-2a, N-methyl imidazole (73 ml), dichloromethane (500 ml) and benzoyl chloride (78 ml) are stirred at 25-30° C. Distilled off the solvent completely from reaction mixture and cooled down to 25-30° C. Aqueous sodium carbonate solution was added to obtained compound at 25-30° C. and stirred. Methanol and water were added to reaction mixture and stirred. Filtered the solid, washed with water and dried to get the compound of formula-4a. Yield: 94.5 g.

### Example-2: Preparation of Compound of Formula-5a

Mixture of compound of formula-4a (90 g), water (180 ml) and acetic acid (360 ml) was heated to 120-125° C. and stirred. Cooled down the reaction mixture to 25-30° C. and stirred. Filtered the solid and washed with water. Acetone (180 ml) and water (270 ml) were added to obtain solid at 25-30° C., heated to 65-70° C. and stirred. Cooled down the mixture to 25-30° C. and stirred. Filtered the solid, washed with water and dried to get the compound of formula-5a. Yield: 68.5 g.

### Example-3: Preparation of Compound of Formula-6

Mixture of compound of formula-5a (65 g), methanol (195 ml) and sodium methoxide solution (40 ml) stirred at 25-30° C. for complete the reaction. After competition of reaction, the acetic acid solution. Distilled off solvent from the reaction mixture and co-distilled with tetrahydrofuran. Tetrahydrofuran (325 ml) was added to residue at 25-30° C. and stirred. Filtered the solid and washed with tetrahydrofuran. Distilled off solvent and co-distilled with acetone. Acetone (65 ml) and n-heptane (260 ml) mixture was added to residue at 28-30° C. and stirred at the same temperature. Filtered the solid, washed with n-heptane and dried to get the compound of formula-6. Yield: 34.6 g

### Example-4: Preparation of Compound of Formula-3

Compound of formula-6 (34 g) and tetrahydrofuran (270 ml) were taken in autoclave, added Pd/C (13.6 g) and applied hydrogen pressure at 25-30° C. Heated the reaction mixture to 65-70° C. and stirred at the same temperature. The reaction mixture was cooled to 25-30° C., filtered the reaction mixture and washed with tetrahydrofuran. Distilled off solvent from the filtrate and co-distilled with acetone. Acetone (68 ml) was added to residue at 25-30° C. and stirred. Filtered the solid, washed with acetone and dried to get the compound of formula-3. Yield: 28 g.

### Example-5: Preparation of Cedazuridine of Formula-1

Cerium chloride (3.5 g) was added to the solution of compound of formula-3 (5 g) in dichloromethane (55 ml) and methanol (37.5 ml) at 25-30° C. Cooled the reaction mixture to 0-5° C., sodium borohydride (1.78 g) was slowly added and stirred the reaction mixture at the same temperature. Filtered the reaction mixture through hy-flow bed and washed with methanol. Distilled off solvent from the filtrate. Water (20 ml) was added to obtained residue at 25-30° C. and stirred. Filtered the solid, washed with water and dried to get the compound of formula-1. Yield: 1.2 g.

### Example-6: Preparation of Compound of Formula-3

Gemcitabine hydrochloride compound of formula-2a (25 g) and water (500 ml) were taken in autoclave, added Pd/C (12.5 g) and applied hydrogen pressure at 25-30° C. Heated the reaction mixture to 50-55° C. and stirred at the same temperature. The reaction mixture was cooled to 25-30° C., filtered the reaction mixture and washed with water. Distilled off solvent from the filtrate and co-distilled with acetone. Acetone (50 ml) was added to residue at 25-30° C., stirred at the same temperature. To the mixture, n-heptane (50 ml) was added at 25-30° C. and stirred at the same temperature. Filtered the solid, washed with n-heptane and dried to get the compound of formula-3. Yield: 14.2 g.

### Example-7: Preparation of Cedazuridine of Formula-1

Cerium chloride heptahydrate (7 g) was added to the solution of compound of formula-3 (10 g) in dichloromethane (110 ml) and methanol (75 ml) at 25-30° C. Cooled the reaction mixture to 0-5° C., sodium borohydride (1.78 g) was slowly added and stirred the reaction mixture at the same temperature for completion of reaction. Filtered the reaction mixture through hy-flow bed and washed with methanol. Distilled off solvent from the filtrate. Water was added to obtained residue at 25-30° C. and stirred at the same temperature. Filtered the solid, washed with water and dried to get the compound of formula-1. Yield: 3.6 g.

### Example-8: Preparation of Compound of Formula-3

Compound of formula-2a (50 g) and tetrahydrofuran (150 ml) and water (100 ml) were taken in autoclave, added Pd/C (25 g) and applied hydrogen pressure at 25-30° C. Heated the reaction mixture to 55-60° C. and stirred at the same temperature. The reaction mixture was cooled to 25-30° C., filtered the reaction mixture and washed with tetrahydrofuran and water. Distilled off solvent from the filtrate and co-distilled with tetrahydrofuran followed by isopropyl alcohol. Isopropyl alcohol was added to residue at 25-30° C., heated to 70-75° C., and stirred. Filtered the unwanted solid, washed with isopropyl alcohol and distilled off the solvent from filtrate. Isopropyl alcohol was added to the obtained compound at 25-30° C., heated to 60° C. and stirred. Cooled to 0° C. to −1° C. and stirred. Filtered the obtained compound, washed with isopropyl alcohol and dried to get the compound of formula-3. Yield: 36 g.

### Example-9: Preparation of Cedazuridine of Formula-1

Cerium chloride heptahydrate (80.5 g) is added to pre-cooled solution of Compound of Formula-3 (115 g) in dichloromethane (1265 ml) and methanol (862 ml) at −11° C. to −9° C. and is added. Sodium borohydride (32.8 g) is added slowly to the above mixture and stirred at −11° C. to −9° C. The reaction mixture is filtered through hy-flow bed, washed with mixture of dichloromethane and methanol and distilled off the half quantity of solvent from the filtrate. To the distillate, activated charcoal was added, stirred at ambient temperature and filtered through hy-flow, washed with mixture of dichloromethane and methanol. The filtrates is distilled and water was added, stirred at ambient temperature. Filtered the solid and dried to get the title compound.

Yield: 66 g. Purity by HPLC: 94% [Pure Cedazuridine]; 5.6% [Epimer].

### Example-10: Purification of Cedazuridine of Formula-1

To the Cedazuridine compound (39 g), acetonitrile (195 ml) and water (19.5 ml) were added and stirred at room temperature. Heated the above mixture to 45° C.-50° C. and stirred. Cooled the mixture to 0-5° C. and stirred. Filtered the solid, washed with acetonitrile and dried. Yield: 35.0 g, Purity by HPLC: 98.53% [Pure Cedazuridine], 1.00% [Epimer].

### Example-11: Purification of Cedazuridine of Formula-1

Mixture of Cedazuridine of formula-1 (16 g), acetone (80 ml) and water (8 ml) is heated to 45° C. The mixture is cooled to room temperature, stirred and filtered the solid and washed with acetone. Dried the obtained solid under vacuum to get the pure compound of formula-1. Yield: 11.0 g; Purity by HPLC: 98.95% [Pure Cedazuridine], 0.9% [Epimer].

### Example-12: Purification of Cedazuridine of Formula-1

Mixture of water (8 ml) and methanol (8 ml) is added to the mixture of Cedazuridine of formula-1 (16 g) and acetonitrile (80 ml) at room temperature. The mixture is heated to 45° C. and stirred at 44-45° C. Cooled the mixture to room temperature, stirred and filtered the solid and washed with acetonitrile. Dried the obtained solid to get the pure compound of formula-1.

Yield: 12.50 g; Purity by HPLC: 99.17% [Pure Cedazuridine], 0.71% [Epimer].

We claim:

1. A process for the preparation of Cedazuridine of Formula-1, comprising: 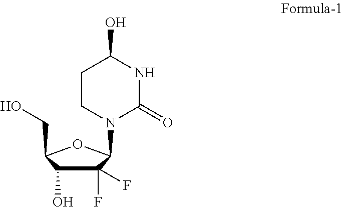
a) reduction of compound of Formula-2 or its hydrochloride salt of Formula-2a 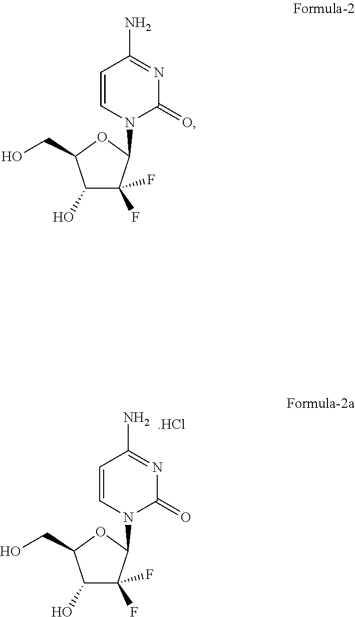
with a reducing agent in presence of hydrogen gas and in a solvent to get the compound of Formula-3 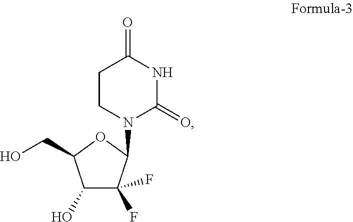
b) reduction of the compound of Formula-3 with reducing agent in presence of a catalyst in a solvent to get the mixture of Cedazuridine of Formula-1 and its epimer;
c) isolating pure Cedazuridine of Formula-1 from the compound obtained in step-b).

2. The process as claimed in claim 1, wherein reducing agent used in step-a) is selected from Platinum, Palladium, Rhodium, Rhenium, Ruthenium, Nickel and Iridium including their oxides, hydroxides, acetates and combinations.

3. The process as claimed in claim 1, wherein the solvent used is step-a) & b) is selected from hydrocarbon solvents, ether solvents, ester solvents, polar-aprotic solvents, chloro solvents, ketone solvents, nitrile solvents, alcohol solvents, polar solvents such as water and/or their mixtures thereof.

4. The process as claimed in claim 1, the isolation of Cedazuridine in step-c) is carried out by suspending the compound obtained in step-b) in a solvent followed by decanting or filtering the mixture; wherein the solvent is selected from nitrile solvents, alcohol solvents, ketone solvents, polar aprotic solvents and water and/or mixtures thereof.

5. The process as claimed in claim 1, wherein the reducing agent used is step-b) is selected from but not limited to Metal hydride including but not limited to LiAlH₄, NaAlH₄, NaBH₄, KBH₄, mixture of NaBH₄ & acetic acid, mixture of NaBH₄ & trifluoroacetic acid, mixture of NaBH₄ & iodine, mixture of NaBH₄ & trimethylchlorosilane, mixture of NaBH₄ & magnesium chloride, mixture of NaBH₄ & calcium chloride, mixture of NaBH₄ & one of transition metal chlorides, mixture of sodium borohydride BF₃·etherate, sodium cyanoborohydride, sodium triacetoxy borohydride, Aluminium hydride (AlH₃), diisobutylaluminium hydride (DIBAL), Vitride {=Sodium bis(2-methoxyethoxy) aluminum hydride}, Lithium Tri-tert-butoxyaluminum Hydride, Tributyltin Hydride; boranes such as not limited to BH₃-tetrahydrofuran, BH₃-dimethyl sulfide; hydrazine and like; and the catalyst selected from Cerium Chloride or Cerium Chloride heptahydrate.

6. A process for the preparation of pure Cedazuridine of Formula-1 comprising;
a) dissolving or suspending the Cedazuridine or isomeric mixture of Cedazuridine of Formula-1 in a solvent or mixture thereof,
b) isolating the pure Cedazuridine of Formula-1 from the mixture obtained in step-a).

7. The process as claimed in claim 6, wherein the solvent used in step-a) is selected from nitrile solvents, alcohol solvents, ketone solvents, polar aprotic solvents and water and/or mixtures thereof.

8. The process as claimed in claim 6, wherein dissolving or suspending the compound in step-a) is carried out at any suitable temperature such as about 25° C. to about reflux temperature of the solvent used.

9. The process as claimed in claim 6, wherein the isolation in step-b) is carried out by the techniques selected from drying, decanting, filtration, purging, cooling the mixture to lower temperatures to precipitate the solid followed by filtration of the mixture, crystallization or crystallization by adding an anti-solvent.
